(19) United States
(12) Patent Application Publication
(10) Pub. No.: US 2023/0201414 A1
(43) Pub. Date: Jun. 29, 2023

(54) DEVICES COMPRISING HYALURONIC ACID AND SILK FIBROIN

(71) Applicant: UNIVERSITY OF MONTANA, Missoula, MT (US)

(72) Inventor: Monica SERBAN, Missoula, MT (US)

(21) Appl. No.: 17/927,037

(22) PCT Filed: May 28, 2021
(86) PCT No.: PCT/US2021/034805

§ 371 Date: Nov. 22, 2022

Related U.S. Application Data
(60) Provisional application No. 63/030,952, filed on May 28, 2020.

(51) Int. Cl. A61L 26/00

(52) U.S. Cl. CPC A61L 26/0023; A61L 26/0028; A61L 2300/20; A61L 2300/412; A61L 2300/60

(57) ABSTRACT

The present disclosure provides biocompatible and bioresorbable devices for tissue repair and regeneration and delivery of an active agent across a biological barrier comprising silk fibroin and hyaluronic acid.

### CROSS-REFERENCE TO RELATED APPLICATIONS

This application claims the benefit of U.S. Provisional Application No, 63/030,952, filed May 28, 2020, the contents of which is herein incorporated by reference in its entirety.

### FIELD

The present invention relates to biocompatible and bioresorbable devices for tissue repair and regeneration and delivery of an active agent across a biological barrier comprising silk fibroin and hyaluronic acid.

### BACKGROUND

Repair and regeneration of damaged and diseased tissues is one of the most complex biological processes that occurs in living organisms. Poor wound healing after trauma, surgery, acute illness, or chronic disease conditions affects millions of humans worldwide each year and is the consequence of poorly regulated elements of the healthy tissue repair response, including inflammation, angiogenesis, matrix deposition, and cell recruitment. Evidence of treating damaged and diseased tissue has found even in the very early stages of human civilization, e.g., by making plasters and bandaging the tissue. Modern advancements in tissue repair include more effective treatment strategies including wound debridement, compression bandaging, wound dressings, hyperbaric oxygen therapy, ultrasound, electrical stimulation, and implantable controlled release drug depots. However, most of these methods rarely interact with endogenous tissue repair and regeneration mechanisms and some require expensive equipment or devices only suitable for hospital settings.

### SUMMARY

Provided herein are biocompatible devices comprising a base layer comprising hyaluronic acid or a combination of silk fibroin and hyaluronic acid. In some embodiments, the base layer is from about 0.5 mm to about 4 mm thick. In some embodiments, the device is about 0.5 mm to about 5 mm thick.

In some embodiments, the hyaluronic acid is conjugated with at least one antioxidant (e.g., methionine, cysteine, tryptophan, tyrosine, homocysteine, Vitamin A, Vitamin C, Vitamin E, or a combination thereof).

In some embodiments, the silk fibroin is chemically or chemoenzymatically modified (e.g., carboxylated silk fibroin, hydroxylated silk fibroin, methylated silk fibroin, diazonium coupled silk fibroin, methacrylated silk fibroin, a silk fibroin modified at a tyrosine, hydroxy, or amine group, or combinations thereof).

In some embodiments, the base layer comprises from about 0% to about 20% w/v silk fibroin. In some embodiments, the base layer comprises from about 0.1% to about 10% w/v hyaluronic acid.

In some embodiments, the base layer further comprises a bioactive macromolecule selected from the group consisting of collagen, gelatin, fibrinogen, elastin, laminin, keratin, actin, myosin, cellulose, amylose, dextran, chitin, glycosaminoglycans, and combinations thereof.

In some embodiments, the device further comprises a microneedle layer comprising a plurality of microneedles comprising a biocompatible material (e.g., silk fibroin, hyaluronic acid, polyglycolic acid (PGA), polylactic acid (PLA), polydioxanone (PDS), polycaprolactone (PCL), poly(lactic-co-glycolic acid) PLGA, polyhydroxyalkanoate (PHA), and conjugates, variants, and combinations thereof).

In some embodiments, the device is self-adherent. In some embodiments, the device further comprises an adhesive layer.

In some embodiments, the device further comprises an active agent. In some embodiments, the active agent is applied to an external surface of the microneedles. In some embodiments, the active agent is embedded throughout the base layer, the microneedle layer, or a combination thereof.

Also provided herein are methods of regenerating and repairing a tissue and methods of delivery an active agent across a biological barrier comprising applying the disclosed devices to a tissue or biological barrier of interest.

Further disclosed are methods of fabricating the disclosed devices. In some embodiments, the methods comprise at least one or all of: preparing a solution of the base layer components comprising hyaluronic acid or a combination of silk fibroin and hyaluronic acid; filling a base layer mold with the solution of the base layer components; removing a solidified base layer from the base layer mold; crosslinking the base layer; filling a microneedle layer mold with a solution of the biocompatible material; removing a solidified microneedle layer from the base layer mold; applying a solution of silk fibroin to one side of the base layer and or the microneedle layer; joining the base layer to the microneedle layer; and adding an active agent. In some embodiments, the methods comprise at least one or all of: preparing a solution comprising hyaluronic acid or a combination of silk fibroin and hyaluronic acid; filling a combined microneedle-base layer mold with the solution; removing a solidified microneedle-base layer from the combined microneedle-base layer mold; crosslinking the solidified microneedle-base layer; and adding an active agent.

Other aspects and embodiments of the disclosure will be apparent in light of the following detailed description and accompanying figures.

### BRIEF DESCRIPTION OF THE DRAWINGS

FIG. 1 is a depiction of a bioengineered system for dermal applications. Inset—Image of an exemplary bioengineered device with a circular microneedle layer.

FIGS. 2A-2C are images of the appearance and flexibility of the epidermal bioengineered base later (FIGS. 2A and 2B) and the device having the appearance and coloration as the skin upon application (FIG. 2C).

FIG. 3 is a graph of the effect of silk fibroin solution concentration and ethanol processing duration of sample mechanical strength.

FIGS. 4A and 4B are graphs of ultimate tensile strength of silk base layers (FIG. 4A; mean±SD, n=4/group) and elastic modulus of silk base layers at 0.25% strain (FIG. 4B; data calculated based on setting 0% strain when 0.05N of initial stress is achieved; data represent mean±SD, n=4/group).

FIG. 5 is a graph of the effect of antioxidant conjugated hyaluronic acid (HAO) on human dermal fibroblast metabolic activity. The data is shown for D-methionine conjugated hyaluronic acid.

FIGS. 6A and 6B are images of histological evaluation (hematoxylin and eosin stained) of fibroblast ingrowth on silk fibroin only (FIG. 6A) and silk fibroin+antioxidant conjugated hyaluronic acid (FIG. 6B) base layers.

FIG. 7 is a graph of representative shear strength for chemically modified silk (carboxylated) at 90 minutes adhesion time.

FIGS. 8A and 8B are images of an exemplary device surface at 450× (FIG. 8A) and 1500× (FIG. 8B) magnification showing through-pores and partial pores.

FIGS. 9A-9B are scanning electron microscope mages of microneedle layers produced from silk fibroin at 50× (FIG. 9A) and 300× (FIG. 9B) magnification. FIG. 9C is an incident light image of an exemplary microneedle device at 30× magnification (FIG. 9C).

FIG. 10 is an image of the microneedle layer loaded with a fluorescent model drug (fluorescein).

FIG. 11 is a graph of the diffusion of a model drug (fluorescein) through the base layer after application onto the device surface.

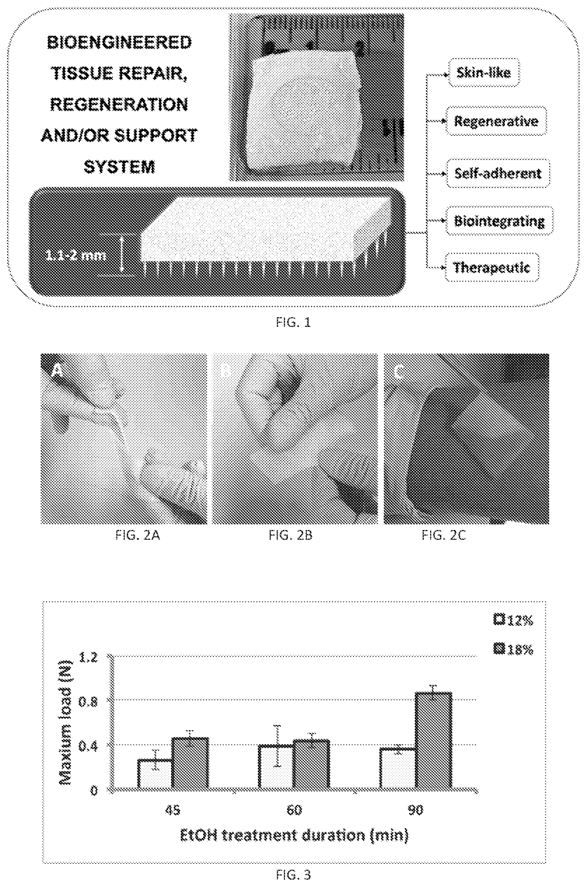

### DETAILED DESCRIPTION OF THE INVENTION

The present disclosure is directed to device formulated from natural biomaterials to feel and look like native tissue for use in regeneration in repair of the tissue and drug delivery to the tissue or underlying areas. The devices are biocompatible and fully assimilate as the new tissue regenerates. The devices comprise a base layer, with may allow adherence to the desired site without the need for sutures or other fixation methods, and, optionally, a microneedle layer, which facilitates local delivery of active agents (e.g., anesthetics, analgesics, and antibiotics) in the site of interest. As shown herein, the disclosed devices accelerate wound healing and tissue repair and regeneration processes.

### 1. Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “subject” or “patient” may be human or non-human and may include, for example, animal strains or species used as “model systems” for research purposes, such a mouse model as described herein. Likewise, patient may include either adults, juveniles (e.g., children), or infants. Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

The terms “contacting” or “applying” as used herein refers to bring or put in contact, to be in or come into contact, or apply to an area, thus referring to a state or condition of touching or of immediate or local proximity.

As used herein, the terms “providing,” “administering,” “introducing,” are used interchangeably herein and refer to the placement of the disclosed device into a subject which results in at least partial localization of the device to a desired site.

As used herein, “bioresorbable” refers to a material that is susceptible to being chemically or enzymatically broken down into lower molecular weight chemical moieties by reagents and conditions that are naturally present in a biological environment. In an in vivo application, the chemical moieties may be assimilated into human or animal tissue, or otherwise removed from the point of implantation. A bioresorbable material that is “substantially completely” resorbed is highly resorbed (e.g., about 95% resorbed, or about 98% resorbed, or about 99% resorbed, or about 99.9% resorbed, or about 99.99% resorbed), but not completely (i.e., about 100%) resorbed. The disclosed device may be partially, substantially, or completely bioresorbed.

As used herein, “biocompatible” refers to a material that does not elicit an immunological rejection or detrimental effect when it is disposed within an in vivo biological environment. For example, a biological marker indicative of an immune response changes less than about 10%, or less than about 20%, or less than about 25%, or less than about 40%, or less than about 50% from a baseline value when a biocompatible material is implanted into a human or animal.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Devices

Provided herein are devices comprising a base layer comprising: hyaluronic acid or a combination of silk fibroin and hyaluronic acid for use in tissue regeneration and delivery of active agents to a tissue of interest.

The devices disclosed herein are preferably biodegradable, biocompatible, and/or bioresorbable. In some embodiments, the device can entirely, substantially, or partially assimilate into a tissue for example, after 1-3 days, 1-3 weeks, 1-3 months or intermediate or greater periods. The desired duration may depend, for example, on the tissues involved and/or the composition of the device. In some embodiments, the device comprises a bioresorbable portion and a non-bioresorbable portion.

The devices may have a wide variety of sizes and shapes based on the intended tissue and area of coverage. The devices preferably are, however, fairly thin to avoid unnecessary bulk when placed on or over tissues. The device may be about 0.5 mm to about 5 mm thick. In some embodiments, the device is about 0.5 mm thick, about 1 mm thick, about 1.5 mm thick, about 2 mm thick, about 2.5 mm thick, about 3 mm thick, about 3.5 mm thick, about 4 mm thick, about 4.5 mm thick, or about 5 mm thick.

Given the devices may be used topically on an exterior tissue surface, the devices preferably are substantially or entirely transparent during usage, or, alternatively or in addition, upon contact with the tissue (e.g., skin) have an appearance similar to that of the tissue. Furthermore, the devices preferably have a level of flexibility consistent with the intended tissue or site of use.

In some embodiments, the base layer comprises hyaluronic acid (HA). Hyaluronic acid, also known as hyaluronan or hyaluronate, is an anionic, non-sulfated glycosaminoglycan polymer comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronic acid can be purified from animal and non-animal sources. Polymers of hyaluronan can range in size from about 1,000 Da to about 20,000,000 Da. Any hyaluronic acid, or pharmaceutically acceptable salt thereof, is useful in the devices disclosed herein. Non-limiting examples of pharmaceutically acceptable salts of hyaluronic acid include sodium hyaluronan, potassium hyaluronan, magnesium hyaluronan, calcium hyaluronan, and combinations thereof.

In some embodiments, the hyaluronic acid is conjugated (e.g., by a covalent bond) with at least one antioxidant. See for example, Kaderli, S., et al., Eur J Pharm Biopharm 2015;90:70-9.

The at least one antioxidant may include any antioxidant known in the art. Antioxidants include any man-made or natural substance which prevents or delays oxidative damage. Examples of antioxidants include, but are not limited to, vitamin A, vitamin C, vitamin E, selenium, and carotenoids (e.g., beta-carotene, lycopene, lutein, and zeaxanthin), glutathione, tocopherols, ubiquinol (coenzyme Q), amino acids (e.g., methionine, cysteine, tryptophan, tyrosine, homocysteine) or any combinations thereof. In some embodiments, the antioxidant comprises methionine, cysteine, tryptophan, tyrosine, homocysteine, Vitamin A, Vitamin C, Vitamin E, or a combination thereof. In particular embodiments, the antioxidant comprises methionine.

The hyaluronic acid may be of any molecular weight. The hyaluronic acid may be 1 kDa to 2 MDa In some embodiments, the hyaluronic acid may have a molecular weight less than about 250 kDa. The hyaluronic acid may have a molecular weight less than about 150 kDa, less than about 100 kDa, less than about 75 kDa, less than about 50 kDa, less than about 25 kDa, less than about 10 kDa, less than about 5 kDa or less than about 1 kDa. The hyaluronic acid may have a molecule weight greater than about 1 kDa, greater than about 5 kDa, greater than about 10 kDa, greater than about 25 kDa, greater than about 50 kDa, greater than about 75 kDa, greater than about 100 kDa, greater than about 150 kDa, or greater than about 200 kDa.

In some embodiments, the hyaluronic acid is high molecular weight hyaluronic acid. High molecular weight hyaluronic acid has a molecular weight greater than about 500 kDa. For example, high molecular weight hyaluronic acid may have a molecular weight between about 500 kDa and about 10 MDa. The hyaluronic acid may have a molecular weight less than 10 MDa, less than 8 MDa, less than 6 MDa, less than 4 MDa, less than 2 MDa, less than 1 MDa, less than 800 kDa, less than 700 kDa, less than 600 kDa, or less than 500 kDa. The hyaluronic acid may have a molecular weight greater than 500 kDa, greater than 600 kDa, greater than 700 kDa, greater than 800 kDa, greater than 900 kDa, greater than 1 MDa, greater than 2 MDa, greater than 4 MDa, greater than 6 MDa, or greater than 8 MDa.

The base layer may comprise from about 0.1% to about 10% w/v hyaluronic acid (HA). In some embodiments, the base layer comprises from about 0.1% w/v HA, about 0.5% w/v HA, about 1% w/v HA, about 2% w/v HA, about 3% w/v HA, about 4% w/v HA, about 5% w/v HA, about 6% w/v HA, about 7% w/v HA, about 8% w/v HA, about 9% w/v HA, or about 10% w/v HA.

In some embodiments, the base layer comprises from about 0.1% to about 0.5% w/v HA, about 0.1% to about 1% w/v HA, about 0.1% to about 2% w/v HA, about 0.1% to about 3% w/v HA, about 0.1% to about 4% w/v HA, about 0.1% to about 5% w/v HA, about 0.1% to about 6% w/v HA, about 0.1% to about 7% w/v HA, about 0.1% to about 8% w/v HA, about 0.1% to about 9% w/v HA, about 0.5% to about 1% w/v HA, about 0.5% to about 2% w/v HA, about 0.5% to about 3% w/v HA, about 0.5% to about 4% w/v HA, about 0.5% to about 5% w/v HA, about 0.5% to about 6% w/v HA, about 0.5% to about 7% w/v HA, about 0.5% to about 8% w/v HA, about 0.5% to about 9% w/v HA, about 0.5% to about 10% w/v HA, about 1% to about 2% w/v HA, about 1% to about 3% w/v HA, about 1% to about 4% w/v HA, about 1% to about 5% w/v HA, about 1% to about 6% w/v HA, about 1% to about 7% w/v HA, about 1% to about 8% w/v HA, about 1% to about 9% w/v HA, about 1% to about 10% w/v HA, about 2% to about 3% w/v HA, about 2% to about 4% w/v HA, about 2% to about 5% w/v HA, about 2% to about 6% w/v HA, about 2% to about 7% w/v HA, about 2% to about 8% w/v HA, about 2% to about 9% w/v HA, about 2% to about 10% w/v HA, about 3% to about 4% w/v HA, about 3% to about 5% w/v HA, about 3% to about 6% w/v HA, about 3% to about 7% w/v HA, about 3% to about 8% w/v HA, about 3% to about 9% w/v HA, about 3% to about 10% w/v HA, about 4% to about 5% w/v HA, about 4% to about 6% w/v HA, about 4% to about 7% w/v HA, about 4% to about 8% w/v HA, about 4% to about 9% w/v HA, about 4% to about 10% w/v HA, about 5% to about 6% w/v HA, about 5% to about 7% w/v HA, about 5% to about 8% w/v HA, about 5% to about 9% w/v HA, about 5% to about 10% w/v HA, about 6% to about 7% w/v HA, about 6% to about 8% w/v HA, about 6% to about 9% w/v HA, about 6% to about 10% w/v HA, about 7% to about 8% w/v HA, about 7% to about 9% w/v HA, about 7% to about 10% w/v HA, about 8% to about 9% w/v HA, about 8% to about 10% w/v HA, or about 9% to about 10% w/v HA.

The base layer may comprise a combination of hyaluronic acid and silk fibroin (SF). The descriptions above for hyaluronic acid conjugation, molecular weight, and percentage are also suitable in the base layer comprising hyaluronic acid and silk fibroin.

In some embodiments, the silk fibroin is chemically or chemoenzymatically modified. A variety of chemical and chemoenzymatic modification of silk fibroin are known in the art. See for example, Kim S H, et al., Nat Commun 2018;9:1620, Simmons L. Tsuchiya K, Numata K. RSC Adv 2016;6:28737-44, and Chen J, Venkatesan H, Hu J Adv Eng Mater 2018;20:1700961. Any of the known modifications may be suitable for use in the devices disclosed herein. In some embodiments, the chemically or chemoenzymatically modified silk fibroin comprises: carboxylated silk fibroin; hydroxylated silk fibroin; methylated silk fibroin; diazonium coupled silk fibroin; methacrylated silk fibroin; a silk fibroin modified at a tyrosine, hydroxy, or amine group or combinations thereof.

The base layer may comprise about 0% silk fibroin (SF). In some embodiments, the base layer comprises from about 0.1 to about 20% w/v silk fibroin. The base layer may comprise about 0.1% w/v, about 0.5% w/v, about 1% w/v, about 5% w/v, about 10% w/v, about 15% w/v, or about 20% w/v SF. In some embodiments, the base layer comprises from about 0.1% to about 0.5% w/v, about 0.1% to about 1% w/v, about 0.1% to about 5% w/v, about 0.1% to about 10% w/v, about 0.1% to about 15% w/v, about 0.5% to about 1% w/v, about 0.5% to about 5% w/v, about 0.5% to about 10% w/v, about 0.5% to about 15% w/v, about 0.5% to about 20% w/v, about 1% to about 5% w/v, about 1% to about 10% w/v, about 1% to about 15% w/v, about 1% to about 20% w/v, about 5% to about 10% w/v, about 5% to about 15% w/v, about 5% to about 20% w/v, about 10% to about 15% w/v, about 10% to about 20% w/v, or about 15% to about 20% w/v SF.

The base layer may further comprise other biocompatible and/or bioresorbable materials, including, but not limited to polytetrafluoroethylene (PTFE), polyurethane, polysulfone, cellulose and variants thereof, polyethylene, polypropylene, polyamide, polyester, polymethylmethacrylate, polylactic acid (PLA), polyglycolic acid (PGA), poly(lactic-co-glycolic acid) (PLGA), hydroxyapatite, polydioxanone (PDS), polycaprolactone (PCL), polyhydroxyalkanoate (PHA), polyglycerol sebacate (PGS), collagen, rice paper, and agarose or other hydrogels.

In some embodiments, the base layer further comprises a bioactive macromolecule. The bioactive macromolecule may comprise collagen, gelatin, fibrinogen, elastin, laminin, keratin, actin, myosin, cellulose, amylose, dextran, chitin, glycosaminoglycans, and combinations thereof.

In some embodiments, the base layer is from about 0.5 mm to about 4 mm thick. The base layer may be about 0.5 mm thick, about 1 mm thick, about 1.5 mm thick, about 2 mm thick, about 2.5 mm thick, about 3 mm thick, about 3.5 mm thick or about 4 mm thick.

In some embodiments, the base layer is self-adherent.

In some embodiments, the device further comprises an adhesive layer. The adhesive layer may comprise any known biocompatible adhesive, including, but not limited to, polymeric adhesives, such as, polyacrylate polymers, rubber-based adhesives, and polysiloxane adhesives. In some embodiments, the adhesive layer comprises silk fibroin.

### a) Microneedle Layer

The device may further comprise a microneedle layer comprising a plurality of microneedles comprising a biocompatible material.

The microneedles may have any shape and/or dimension suitable for insertion into a tissue or across a biological barrier (e.g., skin). For example, the microneedles may fully or completely insert into the tissue, or a portion of the microneedle can be uninserted.

The microneedles may be from about 100 μm to about 2 mm in length. In some embodiments, the microneedles are about 100 μm, about 200 μm, about 300 μm, about 400 μm, about 500 μm, about 600 μm, about 700 μm, about 800 μm, about 900 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm in length.

Each microneedle on the device need not have the same shape and/or dimension. In some embodiments, each microneedle is the same shape and/or dimension. In some embodiments, the device comprises two or more different types of microneedles, each having a defined shape or dimension.

In some embodiments, at least a portion or all of the microneedles are hollow. In some embodiments, at least a portion or all of the microneedles are at least partially filled.

The microneedles may be arranged in any pattern within the microneedle layer and the distance separating each microneedle in the microneedle layer may be varied based on the intended tissue or biological barrier target application.

The biocompatible material may include any of those materials known in the art, including those described and exemplified elsewhere herein. In some embodiments, the biocompatible material is selected from silk fibroin, hyaluronic acid, polyglycolic acid (PGA), polylactic acid (PLA), polydioxanone (PDS), polycaprolactone (PCL), poly(lactic-co-glycolic acid) PLGA, polyhydroxyalkanoate (PHA), and conjugates, variants, and combinations thereof. In some embodiments, the composition of the microneedle is the same or substantially the same as that of the base layer.

### b) Active Agent

The device may further comprise at least one active agent. The active agent may be a small molecule, a protein, an enzyme, a nucleic acid, a hormone, a steroid, an analgesic, an anesthetic, a vitamin, an antimicrobial agent, an anti-inflammatory agent, an antibody, or a combination thereof. In some embodiments, the active agent comprises an anesthetic, an analgesic, an antibiotic, or a combination thereof.

The active agent may be included throughout the device or in localized areas of the device. In some embodiments, the active agent is applied to an external surface of the microneedles. In some embodiments, the active agent is embedded throughout the base layer, the microneedle layer, or a combination thereof. For example, the active agent may diffuse through the pores of the base layer and or the microneedles to access the underlying tissue.

### c) Methods of Fabricating

Also disclosed herein are methods of manufacturing the devices described herein. The devices can be fabricated using a number of methods known in the art including micromachining, lithography, etching, three-dimensional printing, molding methods, or any combination of methods thereof or other methods known in the art for fabrication of similar devices. In some embodiments, the base layer may be fabricated using a different method from that of the microneedle device and/or the adhesive layer, when present. In some embodiments, the base layer and the microneedle device are fabricated using the same methods. In some embodiments, the base layer and the microneedle device are fabricated as one unit using a single method.

In some embodiments, the methods comprise at least one or all of: preparing a solution of the base layer components comprising hyaluronic acid or a combination of silk fibroin and hyaluronic acid; filling a base layer mold with the solution of the base layer components; removing a solidified base layer from the base layer mold; and crosslinking the base layer.

In some embodiments, the methods further comprise, filling a microneedle layer mold with a solution of the biocompatible material; removing a solidified microneedle layer from the base layer mold; and adhering the base layer to the microneedle layer. The adhering can be accomplished using a number of methods know in the art. In some embodiments, the adhering comprises applying a solution of silk fibroin to one side of the base layer and or the microneedle layer; and joining the base layer to the microneedle layer.

In some embodiments, the methods comprise preparing a solution comprising hyaluronic acid or a combination of silk fibroin and hyaluronic acid; filling a combined microneedle-base layer mold with the solution; removing a solidified microneedle-base layer from the combined microneedle-base layer mold; and crosslinking the solidified microneedle-base layer.

The methods may further comprise adding an active agent. In some embodiments, the active agent is added the solution of base layer components and/or the solution of biocompatible material, such that the active agent is homogenously mixed into the base layer, the microneedle layer, or both the base and microneedle layers.

In some embodiments, the active agent is added to the microneedle layer mold prior to filling with the biocompatible material. In some embodiments, the active agent is applied to at least a portion of the microneedle layer mold inner surface.

In some embodiments, the methods comprise forming the base layer and/or the microneedle layer using three-dimensional printing.

In some embodiments, both the base layer and the microneedle layer are formed using three-dimensional printing. In some embodiments, the base layer and the microneedle layer are three-dimensionally printed as a single object. Thus, the microneedle layer is 3D printed directly onto the base layer.

In some embodiments, the base layer or the microneedle layer are formed using a molding process. For example, in some embodiments, the base layer is 3D printed and the microneedle layer is formed in a negative mold. When the microneedle layer is manufacturing using a molding process, an active agent may be added to the mold as described above.

When the base layer and the microneedle layer are formed separately, the two layers can be adhered using methods known in the art. In some embodiments, the two layers are adhered by applying a solution of silk fibroin to one side of the base layer and or the microneedle layer; and joining the base layer to the microneedle layer.

The active agent may be added to the base layer, the microneedle layer, and/or the device after molding and fabrication. In some embodiments, a solution of the active agent is added to the base layer, the microneedle layer, and/or the device. In some embodiments, the active agent is added by applying a solution of the active agent on at least a portion of the base layer, the microneedle layer and/or the device. Thus, the active agent can diffuse into the base layer, the microneedle layer and/or the device due the porous nature of the compositions.

### 3. Methods of Use

### a) Methods of Tissue Regeneration and Repair

The disclosure also provides methods of regenerating and repairing a tissue comprising applying the disclosed devices to the tissue. Tissue regeneration and repair references processes to grow, renew or restore at least a portion of a tissue which has been damaged, lost, or diseased to return the tissue, at least partially, to its original structural, functional and physiological condition. In some embodiments, the tissue comprises a wound, an abrasion or loss of tissue, a burn, a suture, a cut, or any combination thereof.

The devices disclosed herein may modulate cell migration and proliferation, thereby reducing inflammation, accelerating wound healing, reduce scarring and ultimately promote repair, regeneration and restoration of structure and function in the tissue of interest. In some embodiments, the regeneration and repair comprise stimulation of fibroblast ingrowth into the tissue. In some embodiments, the regeneration and repair comprise increasing the metabolic activity of fibroblasts.

The tissue may be a soft tissue, including muscles, fibrous tissues, and fat. In some embodiments, the tissue comprises skin, muscle, fascia, or a subcutaneous tissue.

### b) Methods of Delivery of an Active Agent

The disclosure also provides methods for delivery of an active agent across a biological barrier comprising applying a device as disclosed herein to the biological barrier. A biological barrier can include any barrier to a tissue, including, but not limited to cell membranes, skin or layers thereof (e.g., epidermal and dermal tissue layers), other tissue layers (e.g., mucosal tissues, vascular tissues, and the like). In some embodiments, the biological barrier is the skin. In some embodiments, the biological barrier is the surface of a tissue.

Descriptions of devices and active agents set forth above is applicable to the disclosed methods.

### 4. Kits

Also within the scope of the present disclosure are kits that include devices described herein. In some embodiments, the kits comprise the devices described herein and an active agent. Descriptions of devices and active agents set forth above is also applicable to the kits.

Individual member components of the kits may be physically packaged together or separately. The components of the kit may be provided in bulk packages (e.g., multi-use packages) or single-use packages. The kits can also comprise instructions for using the components of the kit. The instructions are relevant materials or methodologies pertaining to the kit. The materials may include any combination of the following: background information, list of components and their availability information (purchase information, etc.), brief or detailed protocols for using the compositions, troubleshooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain additional containers or devices for use with the methods disclosed herein. The kits optionally may provide additional components such wound dressings (gauze, adhesive bandages and the like), cotton swabs or wipes, and cleaning or antibiotic wipes.

The kits provided herein are in suitable packaging. Suitable packaging includes, but is not limited to, vials, bottles, jars, flexible packaging, and the like.

The following examples further illustrate the invention but should not be construed as in any way limiting its scope.

### EXAMPLES

### Materials and Methods

Hyaluronic Acid (HA)-antioxidant: HA can be used alone or chemically conjugated though various chemistries with antioxidants such as vitamin A, vitamin C, vitamin E, methionine, etc. as known in the art (See, Serban M A, Skardal A. Matrix Biology 2019;78-79:337-45, incorporated herein by reference in its entirety). In a first step, HA was modified with chloroacetic acid to increase the conjugation sites of the polymer. Specifically, 0.8 g of HA were dissolved in 8 mL of 45% w/v NaOH solution and magnetically stirred at room temperature for two hours before adding 60 mL of isopropanol and continuing to stir. Iodoacetic acid (20 mL solution of 0.432 M) was added to the HA/isopropanol mixture before covering with parafilm and stirring for 2 hours at room temperature. This mixture was filtered through a Buechner funnel (#2 Whatman filter paper) and the resulting carboxymethyl-HA (CMHA) cake was dissolved in 80 mL of DI water. The pH of the solution was adjusted to ˜7.0 using 6N HCl. This CMHA solution was subsequently purified by dialysis against DI water (3500 MWCO dialysis cassettes) for 72 hours with 3-4 water changes per day to remove excess iodoacetic acid. The purified CMHA solution was removed from the cassettes, frozen at −80° C. for 3 hours or until fully frozen, and then lyophilized until completely dry. Next, antioxidants were covalently attached to CMHA using carbodiimide chemistry. Specifically, 25 mg of CMHA was dissolved into 5 mL of 2-(N-morpholino) ethanesulfonic acid (MES) buffer in a 50 mL beaker covered in parafilm with stir. The solution was mixed until CMHA was fully dissolved (about 25 minutes) and the antioxidant was added in a 1:2, 1:10 or 1:20 molar ratio, respectively, and allowed to mix until fully dissolved (about 5 minutes). A zero-length crosslinker [(1-ethyl-3-(3-dimethylaminopropyl) carbodiimide or EDC, 50 mg) was then added to the reaction mix and allowed to react for 20 hours. After 20 hours, the reaction was neutralized to a pH of 7.0 with an NaOH solution. The contents of the beaker were then added into dialysis cassettes and dialyzed against DI water, similarly to CMHA.

Formation of SF/HA base layer: A 135-155 mg/mL solution of silk fibroin (SF) was combined with a 40 mg/mL solution of HA (either unmodified or chemically modified with antioxidant) and water to yield a series of varying mixes ranging from 60-120 mg/mL SF and 2-5 mg/mL HA component. A defined volume (2.2. mL) of this mix was cast into a 4° C. pre-chilled 25×75×1 mm polydimethylsiloxane (PDMS) negative mold. This was then placed at −80° C. until frozen, then lyophilized overnight to yield 1.0-1.2 mm thick base layers. These were then submerged in 90% EtOH for 24 hours to induce physical crosslinking then cut to desired size before being allowed to air dry.

Formation of microneedle layer: Microneedle template molds (500 μm needle height, 600 μm needle pitch) were epoxied to a three-dimensional (3D) printed base and used to cast PDMS negatives of the microneedle patch. SF solution alone or mixed with other macromolecules (60-80 mg/mL) was then placed into negative mold and allowed to dry for 24-48 hours at room temperature. The resulting microneedle layer was then submerged in 90% EtOH before allowing to air dry.

Formation of microneedle and base layer assembly: A microneedle layer and a base layer were briefly placed in deionized (DI) water to allow flexing and bending without breaking. A small paintbrush was used to apply a thin layer of 60-80 mg/mL silk fibroin solution to one side of the base layer and the microneedles were adhered to the base layer. This was allowed to air dry completely before crosslinking the silk adhesive for a few hours with 90% EtOH. If alcohol (methanol, ethanol, isopropanol, etc.) treatment was not applied, the assembled system is adherent.

Base layer tension testing: Base layers were cast and crosslinked as described above. A 3D printed dog bone-shaped stencil (40 mm length, 10 mm width in testing region) was used to cut out ˜1 mm thick samples for tension testing on a DHR2 Rheometer. After crosslinking and drying, each tissue was briefly rinsed in DI water before allowing it to soak in phosphate buffered saline (PBS) for 30 min. Excess moisture was then dabbed off with a paper towel before loading the tissue into the tension testing fixture. A 20 mm loading gap was used and widened at a constant rate of 166.667 μm/sec.

### Cell Culture Experiments

MTS assay. Primary adult fibroblasts were seeded at 1.5E4 cells/well in a 96-well plate, allowed to establish for 24 hours, then exposed to respective treatment in growth media for 24 hrs. Cell-titer aqueous one MTS assay was then used to determine metabolic activity relative to control.

CyQuant assay. These assays were set up identically to the above MTS assay, but rather than testing for cellular metabolic activity, cell viability/proliferation (based on the amount of DNA) was determined using the CyQuant NF assay kit.

Histology. Base layers (˜1 mm thick) were cast into cell culture inserts, lyophilized, then crosslinked/sterilized for 24 hours with 90% EtOH. The EtOH was allowed to dry off completely in sterile conditions then each tissue was pre-soaked for 2 hours in fibroblast growth media. Primary adult fibroblasts were seeded at a density of 1E5 on the top of the base layers and allowed to grow submerged in growth media for 14 days before fixing, processing, sectioning, and staining with hematoxylin and eosin (H&E).

### Example 1

Bioengineered systems that mimic the natural skin feel and appearance were made of natural biomaterials such as silk fibroin (SF) and hyaluronic acid (HA) (FIGS. 2A-2C). In addition to SF, natural skin-specific macromolecules such as HA, collagen, elastin, laminin, fibronectin, etc.—either as natural macromolecules or chemically modified—can be incorporated into the SF devices in order to maximize their tissue-like properties.

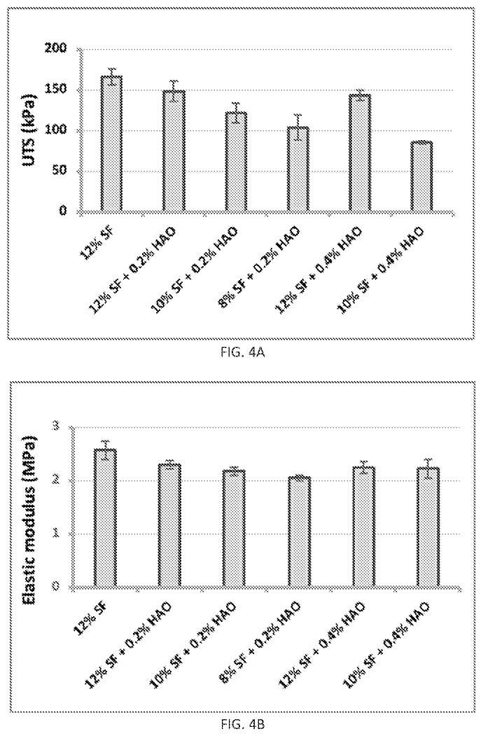

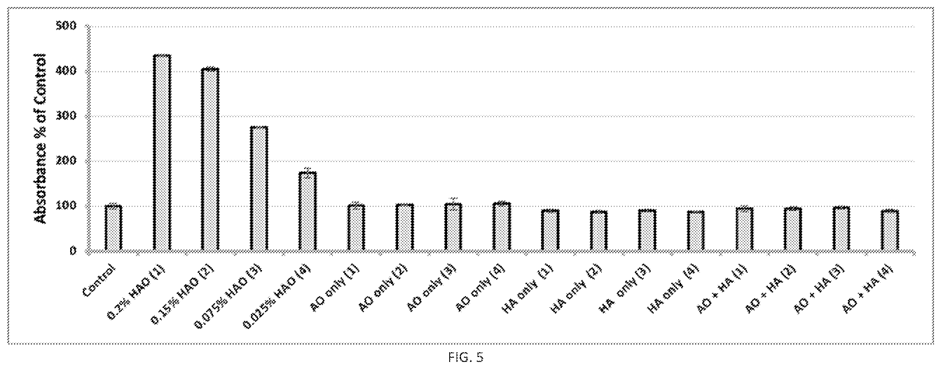

The mechanical properties of silk are partly responsible for its protective effects. By simply changing the biomaterial formulation or by altering the processing parameters, the mechanical properties of the skin like-devices can be tailored. Specifically, from a processing perspective, skin-like devices containing 12% and 18% w/v SF, respectively, were prepared and treated with 90% ethanol (EtOH) between 45-90 minutes. Subsequently samples were subjected to a tension test to evaluate their strength (FIG. 3). The results indicate a significant increase in sample strength with increased SF content and length of EtOH processing. From a formulation perspective, while keeping the processing parameters constant (24 hours 90% EtOH treatment) but varying the material composition, the mechanical properties of the constructs can be further customized (FIGS. 4A-4B).

### Example 2

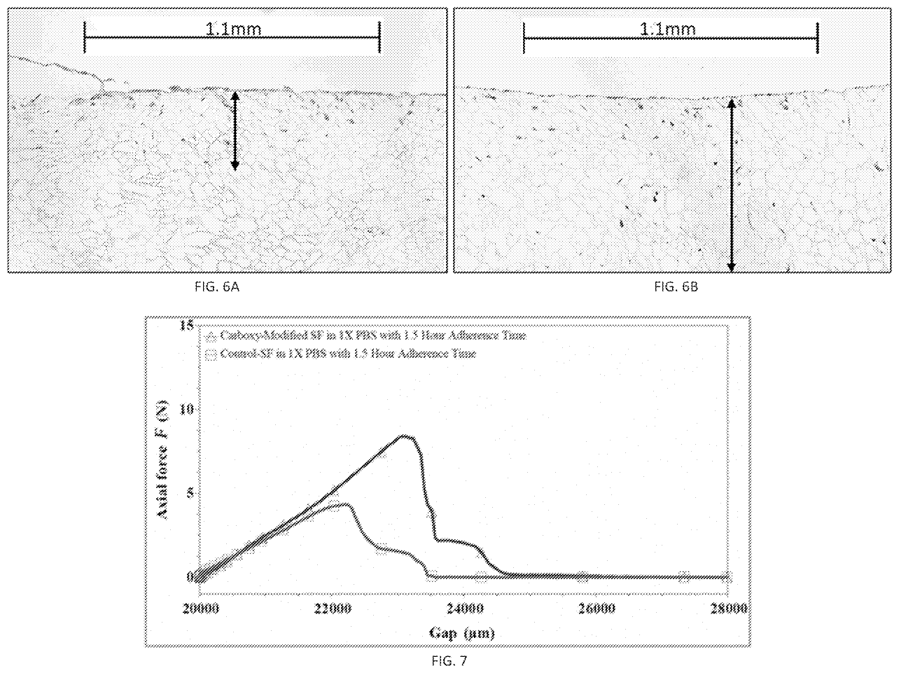

In response to a skin injury, wound healing occurs in three stages: inflammation, cellular proliferation and scar formation, and scar tissue remodeling. By chemically modifying HA with antioxidants to yield HA-antioxidant conjugates (HAO) the metabolic activity of primary human dermal fibroblasts was increased (FIG. 5). Use of antioxidant alone or antioxidant with HA but not conjugated did not produce a similar effect. Moreover, fibroblasts grew into constructs prepared with HAO to a higher extent compared to the SF only constructs (FIG. 6). Overall, these data indicate that exemplary devices, as disclosed herein can enhance wound healing and repair.

### Example 3

Currently available skin substitutes require fixation with sutures or staples and that increases the morbidity to the patient and procedure time for the medical personnel. On-contact adhesives entirely made of SF can be produced from SF solutions. By chemically modifying SF these adhesive properties can be further tailored (FIG. 7).

### Example 4

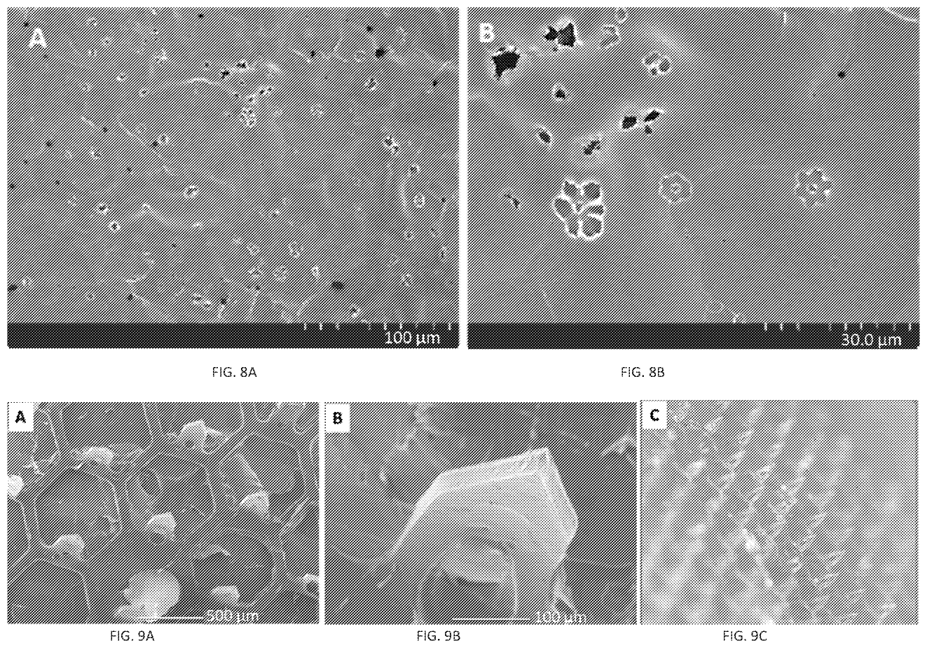

Patients that present with large surface wounds and especially with burns, are commonly treated with pain management medication such as opioids, and antibiotics for infection prevention. Systemic treatment of patients with opioids can lead to tolerance leading to dose escalation and ultimately dependence. The devices disclosed herein enable localized drug delivery to help minimize or eliminate the need for systemic drug administration, without the need for a drug to be actually incorporated into the system. Rather, these drugs would simple diffuse or pass though the bioengineered systems when applied to the surface. The surface architecture of the SF materials described in FIG. 2 was investigated by scanning electron microscopy (SEM). No pore-inducing agents (porogens) were used during the preparation and processing of these materials. The images revealed a micro-porous structure with opening in the range of 5-50 μm (FIG. 8A). Some of the porous structures have a funnel-like structure with one circular, narrower base and one wider, flower-like base (FIG. 8B). The porosity of the devices can be further customized via the use of porogens by following well-established protocols.

### Example 5

As mentioned above, the skin-like devices would, through their porous structure allow the passage of drugs to the underlaying tissues. These drugs could simple be applied as liquids on the surface of the device and would be expected to penetrate the device and reach the tissue. However, for optimal therapeutic effects, such drugs would ideally need to reach the dermis (tissue layer under the epidermis, that contains nerve endings and blood vessels) dermal layer of the skin. Although in many cases of severe injuries the dermis is exposed, the drug deployment properties the bioengineered system can be enhanced by incorporating microneedles into the device design. These microneedles would ensure the delivery of drugs to the dermis regardless of its exposure. By casting silk solutions into microneedle molds, high fidelity biomaterial microneedles were obtained (FIG. 9).

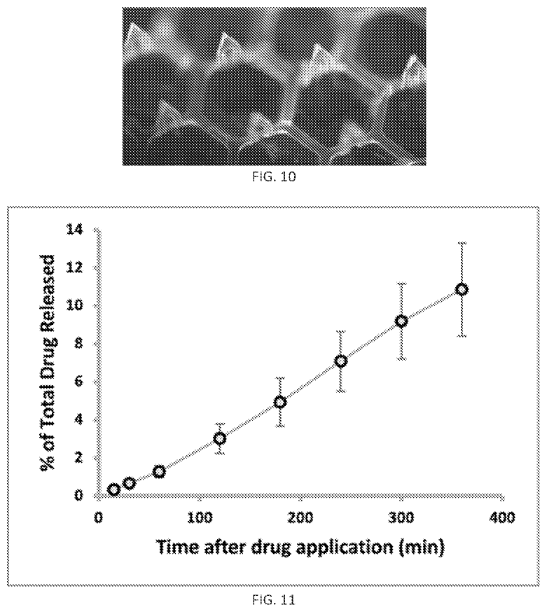

A drug or combination of drugs can be loaded onto or into the device for immediate or slow release upon placement at the wound site. This could be achieved either by placing the drug or drug solution into the microneedle mold, then casting the microneedles to have the drug on the microneedle surface (FIG. 10). Drugs could also be loaded into the base layer during or after formation, or into the microneedles by mixing into the casting solution. FIG. 11 shows the diffusion of fluorescein through the device, when applied onto the base layer. Alternatively, due to the porous structure of the base layer, drugs could be applied onto the device (base layer side) and would diffuse to the other side of the construct.

For reasons of completeness, various aspects of the invention are set out in the following numbered clauses:

Clause 1. A biocompatible device comprising a base layer comprising:
hyaluronic acid; or
a combination of silk fibroin and hyaluronic acid.

Clause 2. The device of clause 1. wherein the hyaluronic acid is conjugated with an antioxidant.

Clause 3. The device of clause 2, wherein the antioxidant comprises methionine, cysteine, tryptophan, tyrosine, homocysteine, Vitamin A, Vitamin C, Vitamin E, or a combination thereof.

Clause 4. The device of any of clauses 1-3, wherein the silk fibroin is chemically or chemoenzymatically modified.

Clause 5. The device of clause 4, wherein the chemically or chemoenzymatically modified silk fibroin comprises: carboxylated silk fibroin; hydroxylated silk fibroin; methylated silk fibroin; diazonium coupled silk fibroin; methacrylated silk fibroin; a silk fibroin modified at a tyrosine, hydroxy, or amine group; or combinations thereof.

Clause 6. The device of any of clauses 1-5, wherein the base layer comprises from about 0% to about 20% w/v silk fibroin.

Clause 7. The device of any of clauses 1-6, wherein the base layer comprises from about 0.1% to about 10% w/v hyaluronic acid.

Clause 8. The device of any of clauses 1-7, wherein the base layer further comprises a bioactive macromolecule selected from the group consisting of collagen, gelatin, fibrinogen, elastin, laminin, keratin, actin, myosin, cellulose, amylose, dextran, chitin, glycosaminoglycans, and combinations thereof.

Clause 9. The device of any of clauses 1-8, wherein the base layer is from about 0.5 mm to about 4 mm thick.

Clause 10. The device of any of clauses 1-9, further comprising a microneedle layer comprising a plurality of microneedles comprising a biocompatible material.

Clause 11. The device of clause 10, wherein the microneedles are hollow.

Clause 12. The device of clause 10, wherein the microneedles are at least partially filled.

Clause 13. The device of any of clauses 10-12, wherein the microneedles are about 100 μm to about 2 mm in length.

Clause 14. The device of any of clauses 10-13, wherein the biocompatible material is selected from silk fibroin, hyaluronic acid, polyglycolic acid (PGA), polylactic acid (PLA), polydioxanone (PDS), polycaprolactone (PCL), poly(lactic-co-glycolic acid) PLGA, polyhydroxyalkanoate (PHA), and conjugates, variants, and combinations thereof.

Clause 15. The device of any of clauses 1-14, wherein the base layer is self-adherent.

Clause 16. The device of any of clauses 1-15, wherein the device further comprises an adhesive layer comprising silk fibroin.

Clause 17. The device of any of clauses 1-16, wherein the device further comprises an active agent.

Clause 18. The device of clause 17, wherein the active agent is applied to an external surface of the microneedles.

Clause 19. The device of clause 17, wherein the active agent is embedded throughout the base layer, the microneedle layer, or a combination thereof.

Clause 20. The device of any of clauses 17-19, wherein the active agent comprises a small molecule, a protein, an enzyme, a nucleic acid, a hormone, a steroid, an analgesic, an anesthetic, a vitamin, an antimicrobial agent, an anti-inflammatory agent, an antibody, or a combination thereof.

Clause 21. The device of any of clauses 17-20, wherein the active agent comprises an anesthetic, an analgesic, an antibiotic, or a combination thereof.

Clause 22. The device of any of clauses 1-21, wherein the device is about 0.5 mm to about 5 mm thick.

Clause 23. A kit comprising a device of any of clauses 1-16 and an active agent.

Clause 24. A method of regenerating and repairing a tissue comprising applying the device of any of clauses 1-22 to the tissue.

Clause 25. The method of clause 24, wherein the tissue comprises a wound, an abrasion or loss of tissue, a burn, a suture, a cut, or any combination thereof.

Clause 26. The method of clause 24 or 25, wherein the tissue comprises skin, muscle, fascia, or a subcutaneous tissue.

Clause 27. The method of any of clauses 24-26, wherein the regenerating and repairing a tissue comprises stimulating fibroblast ingrowth into the tissue and increasing the metabolic activity of fibroblasts.

Clause 28. A method of delivery an active agent across a biological barrier comprising: applying the device of any of clauses 1-22 to the biological barrier.

Clause 29. The method of clause 28, wherein the biological barrier is the skin.

Clause 30. The method of clause 29, wherein the biological barrier is the surface of a tissue.

Clause 31. A method of fabricating the device of any of clauses 1-21, the method comprising:
preparing a solution of the base layer components comprising hyaluronic acid or a combination of silk fibroin and hyaluronic acid;
filling a base layer mold with the solution of the base layer components;
removing a solidified base layer from the base layer mold; and
crosslinking the base layer.

Clause 32. The method of clause 31, further comprising:
filling a microneedle layer mold with a solution of the biocompatible material;
removing a solidified microneedle layer from the base layer mold;
applying a solution of silk fibroin to one side of the base layer and or the microneedle layer; and
joining the base layer to the microneedle layer.

Clause 33. The method of clause 32, wherein the method further comprises adding an active agent to the microneedle layer mold prior to filling with the biocompatible material, adding the active agent to the biocompatible material, or a combination thereof.

Clause 34. The method of clause 33, wherein adding the active agent to the microneedle layer mold comprises applying the active agent to a least a portion of the microneedle layer mold inner surface.

Clause 35. The method of any of clauses 31-34, wherein the method further comprises adding an active agent to the base layer, the microneedle layer, the device, or any combination thereof.

Clause 36. The method of clause 35, wherein adding the active agent comprises soaking the base layer, the microneedle layer, the device, or any combination thereof with a solution of the active agent or pouring a solution of the active agent on the base layer, the microneedle layer, the device, or any combination thereof.

Clause 37. A method of fabricating the device of any of clauses 10-22, the method comprising:
preparing a solution comprising hyaluronic acid or a combination of silk fibroin and hyaluronic acid;
filling a combined microneedle-base layer mold with the solution;
removing a solidified microneedle-base layer from the combined microneedle-base layer mold; and
crosslinking the solidified microneedle-base layer.

Clause 38. The method of clause 37, wherein the method further comprises adding an active agent to the combined microneedle-base layer mold prior to filling with the solution.

Clause 39. The method of clause 38, wherein adding the active agent to the combined microneedle-base layer mold comprises applying the active agent to at least a portion of the combined microneedle-base layer mold inner surface.

Clause 40. The method of any of clauses 37-39, wherein the method further comprises adding an active agent to the device.

Clause 41. The method of clause 40, wherein adding the active agent comprises soaking the device with a solution of the active agent or pouring a solution of the active agent on the device.

Clause 42. A method of fabricating the device of any of clauses 10-22, the method comprising:
forming the base layer using three-dimensional printing.

Clause 43. The method of clause 42, further comprising preparing a microneedle layer.

Clause 44. The method of clause 43, wherein the microneedle layer is prepared using a method comprising:
filling a microneedle layer mold with a solution of the biocompatible material; and
removing a solidified microneedle layer from the base layer mold.

Clause 45. The method of clause 43, wherein the microneedle layer is prepared using three-dimensional printing.

Clause 46. The method of any of clauses 43-45, further comprising adhering the microneedle layer to the base layer.

Clause 47. The method of clause 46, wherein the adhering comprises:
applying a solution of silk fibroin to one side of the base layer and or the microneedle layer; and
joining the base layer to the microneedle layer.

Clause 48. The method of clause 46, wherein the adhering comprises 3D printing the microneedle layer directly on the base layer.

Clause 49. The method of clause 43, wherein the method further comprises adding an active agent to the microneedle layer mold prior to filling with the biocompatible material, adding the active agent to the biocompatible material, or a combination thereof.

Clause 50. The method of clause 49, wherein adding the active agent to the microneedle layer mold comprises applying the active agent to a least a portion of the microneedle layer mold inner surface.

Clause 51. The method of any of clauses 42-50, wherein the method further comprises adding an active agent to the base layer, the microneedle layer, the device, or any combination thereof.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A biocompatible device comprising a base layer comprising:
hyaluronic acid; or
a combination of silk fibroin and hyaluronic acid.

2. The device of claim 1, wherein the hyaluronic acid is conjugated with an antioxidant.

3. The device of claim 2, wherein the antioxidant comprises methionine, cysteine, tryptophan, tyrosine, homocysteine, Vitamin A, Vitamin C, Vitamin E, or a combination thereof.

4. The device of any of claims 1-3, wherein the silk fibroin is chemically or chemoenzymatically modified.

5. The device of claim 4, wherein the chemically or chemoenzymatically modified silk fibroin comprises: carboxylated silk fibroin; hydroxylated silk fibroin; methylated silk fibroin; diazonium coupled silk fibroin; methacrylated silk fibroin; a silk fibroin modified at a tyrosine, hydroxy, or amine group; or combinations thereof.

6. The device of any of claims 1-5, wherein the base layer comprises from about 0% to about 20% w/v silk fibroin.

7. The device of any of claims 1-6, wherein the base layer comprises from about 0.1% to about 10% w/v hyaluronic acid.

8. The device of any of claims 1-7, wherein the base layer further comprises a bioactive macromolecule selected from the group consisting of collagen, gelatin, fibrinogen, elastin, laminin, keratin, actin, myosin, cellulose, amylose, dextran, chitin, glycosaminoglycans, and combinations thereof.

9. The device of any of claims 1-8, wherein the base layer is from about 0.5 mm to about 4 mm thick.

10. The device of any of claims 1-9, further comprising a microneedle layer comprising a plurality of microneedles comprising a biocompatible material.

11. The device of claim 10, wherein the microneedles are hollow.

12. The device of claim 10, wherein the microneedles are at least partially filled.

13. The device of any of claims 10-12, wherein the microneedles are about 100 μm to about 2 mm in length.

14. The device of any of claims 10-13, wherein the biocompatible material is selected from silk fibroin, hyaluronic acid, polyglycolic acid (PGA), polylactic acid (PLA), polydioxanone (PDS), polycaprolactone (PCL), poly(lactic-co-glycolic acid) PLGA, polyhydroxyalkanoate (PHA), and conjugates, variants, and combinations thereof.

15. The device of any of claims 1-14, wherein the base layer is self-adherent.

16. The device of any of claims 1-15, wherein the device further comprises an adhesive layer comprising silk fibroin.

17. The device of any of claims 1-16, wherein the device further comprises an active agent.

18. The device of claim 17, wherein the active agent is applied to an external surface of the microneedles.

19. The device of claim 17, wherein the active agent is embedded throughout the base layer, the microneedle layer, or a combination thereof.

20. The device of any of claims 17-19, wherein the active agent comprises a small molecule, a protein, an enzyme, a nucleic acid, a hormone, a steroid, an analgesic, an anesthetic, a vitamin, an antimicrobial agent, an anti-inflammatory agent, an antibody, or a combination thereof.

21. The device of any of claims 17-20, wherein the active agent comprises an anesthetic, an analgesic, an antibiotic, or a combination thereof.

22. The device of any of claims 1-21, wherein the device is about 0.5 mm to about 5 mm thick.

23. A kit comprising a device of any of claims 1-16 and an active agent.

24. A method of regenerating and repairing a tissue comprising applying the device of any of claims 1-22 to the tissue.

25. The method of claim 24, wherein the tissue comprises a wound, an abrasion or loss of tissue, a burn, a suture, a cut, or any combination thereof.

26. The method of claim 24 or 25, wherein the tissue comprises skin, muscle, fascia, or a subcutaneous tissue.

27. The method of any of claims 24-26, wherein the regenerating and repairing a tissue comprises stimulating fibroblast ingrowth into the tissue and increasing the metabolic activity of fibroblasts.

28. A method of delivery an active agent across a biological barrier comprising: applying the device of any of claims 1-22 to the biological barrier.

29. The method of claim 28, wherein the biological barrier is the skin.

30. The method of claim 29, wherein the biological barrier is the surface of a tissue.

31. A method of fabricating the device of any of claims 1-21, the method comprising:
preparing a solution of the base layer components comprising hyaluronic acid or a combination of silk fibroin and hyaluronic acid;
filling a base layer mold with the solution of the base layer components;
removing a solidified base layer from the base layer mold; and
crosslinking the base layer.

32. The method of claim 31, further comprising:
filling a microneedle layer mold with a solution of the biocompatible material;
removing a solidified microneedle layer from the base layer mold;
applying a solution of silk fibroin to one side of the base layer and or the microneedle layer; and
joining the base layer to the microneedle layer.

33. The method of claim 32, wherein the method further comprises adding an active agent to the microneedle layer mold prior to filling with the biocompatible material, adding the active agent to the biocompatible material, or a combination thereof.

34. The method of claim 33, wherein adding the active agent to the microneedle layer mold comprises applying the active agent to a least a portion of the microneedle layer mold inner surface.

35. The method of any of claims 31-34, wherein the method further comprises adding an active agent to the base layer, the microneedle layer, the device, or any combination thereof.

36. The method of claim 35, wherein adding the active agent comprises soaking the base layer, the microneedle layer, the device, or any combination thereof with a solution of the active agent or pouring a solution of the active agent on the base layer, the microneedle layer, the device, or any combination thereof.

37. A method of fabricating the device of any of claims 10-22, the method comprising:
preparing a solution comprising hyaluronic acid or a combination of silk fibroin and hyaluronic acid;
filling a combined microneedle-base layer mold with the solution;
removing a solidified microneedle-base layer from the combined microneedle-base layer mold; and
crosslinking the solidified microneedle-base layer.

38. The method of claim 37, wherein the method further comprises adding an active agent to the combined microneedle-base layer mold prior to filling with the solution.

39. The method of claim 38, wherein adding the active agent to the combined microneedle-base layer mold comprises applying the active agent to at least a portion of the combined microneedle-base layer mold inner surface.

40. The method of any of claims 37-39, wherein the method further comprises adding an active agent to the device.

41. The method of claim 40, wherein adding the active agent comprises soaking the device with a solution of the active agent or pouring a solution of the active agent on the device.

42. A method of fabricating the device of any of claims 10-22, the method comprising:
forming the base layer using three-dimensional printing.

43. The method of claim 42, further comprising preparing a microneedle layer.

44. The method of claim 43, wherein the microneedle layer is prepared using a method comprising:
filling a microneedle layer mold with a solution of the biocompatible material; and
removing a solidified microneedle layer from the base layer mold.

45. The method of claim 43, wherein the microneedle layer is prepared using three-dimensional printing.

46. The method of any of claims 43-45, further comprising adhering the microneedle layer to the base layer.

47. The method of claim 46, wherein the adhering comprises:
applying a solution of silk fibroin to one side of the base layer and or the microneedle layer; and
joining the base layer to the microneedle layer.

48. The method of claim 46, wherein the adhering comprises 3D printing the microneedle layer directly on the base layer.

49. The method of claim 43, wherein the method further comprises adding an active agent to the microneedle layer mold prior to filling with the biocompatible material, adding the active agent to the biocompatible material, or a combination thereof.

50. The method of claim 49, wherein adding the active agent to the microneedle layer mold comprises applying the active agent to a least a portion of the microneedle layer mold inner surface.

51. The method of any of claims 42-50, wherein the method further comprises adding an active agent to the base layer, the microneedle layer, the device, or any combination thereof.
